(19) United States
(12) Patent Application Publication
(10) Pub. No.: US 2017/0264797 A1
(43) Pub. Date: Sep. 14, 2017

(54) DRIVER MONITORING SYSTEM IN A MOTOR VEHICLE

(71) Applicant: ROBBERT BOSCH GMBH, Stuttgart (DE)

(72) Inventors: Hoang TRINH, Gerlingen (DE); Markus LINDNER, Stuttgart (DE)

(73) Assignee: Robert Bosch GmbH, Stuttgart (DE)

(21) Appl. No.: 15/329,844

(22) PCT Filed: Jun. 18, 2015
(86) PCT No.: PCT/EP2015/063735

§ 371 Date: Jan. 27, 2017

(30) Foreign Application Priority Data
Aug. 11, 2014 (DE) 10 2014 215 856.7

(51) Int. Cl. H04N 5/225; G06K 9/20; G06K 9/00; B60R 11/04; B60K 37/02

(52) U.S. Cl. CPC H04N 5/2253; B60R 11/04; B60K 37/02; G06K 9/00845; G06K 9/209; B60R 2011/0005

(57) ABSTRACT

A driver monitoring system for a motor vehicle, including at least one camera device, which has at least one camera unit situated on a support element and at least one infrared emitter. It is provided that the support element includes at least one device for locking the infrared emitter in place on the support element.

### FIELD

The present invention relates to a driver monitoring system in a motor vehicle, including at least one camera device, which has at least one camera unit situated on a support element and at least one infrared emitter.

### BACKGROUND INFORMATION

Conventional driver monitoring systems may include at least one camera device. In order to detect the driver behavior, for example, in order to be able to determine whether the driver is observing the traffic situation or is exhibiting signs of fatigue, conventional camera-based driver monitoring systems watch the face of the driver and establish the behavior of the driver as a function of the detected facial characteristics or of the orientation of the driver's head. In order to still be able to carry out such observations even when the passenger compartment is dark, for example, during night driving, an infrared emitter is provided, which illuminates the driver so that a camera unit of the camera device, in particular, an infrared camera, is able to watch the driver in the dark as well.

The camera unit, which is generally made up of a camera chip and optics assigned to the chip for directing the light in a targeted manner, and the infrared emitter must be advantageously mounted in the motor vehicle in order to be able to reliably monitor the driver. Thus, the integration of the camera device into the motor vehicle may be an important factor in the efficiency of the system.

### SUMMARY

An example driver monitoring system according to the present may have the advantage that the camera device is installable in a particularly compact and therefore modular-like manner, the camera unit and the infrared emitter being present together as a manageable unit, so that an electrical contacting of camera and infrared emitter may also be simply and cost-effectively designed. For this purpose, it is provided according to the present invention that the support element on which the camera unit is normally situated also includes a device for locking the infrared emitter on the support element. Thus, the infrared emitter is affixed on the same support element which also holds the camera unit. This offers a compact unit, which may be easily installed in the motor vehicle. With the shared support element, an electrical contacting of camera and infrared emitter is also easily implementable. Thus, for example, the support element may be provided with a single contact plug or contact terminal, with which both the camera as well as the infrared emitter are electrically contactable. The spatial proximity of camera and infrared emitter also ensures that an optimal driver illumination and driver monitoring takes place.

According to one preferred refinement of the present invention, it is provided that the device is designed for the detachable locking of the infrared emitter. While it is conceivable, in principle, to design the device, for example, in the form of a solder joint or weld joint in order to provide a permanent locking of the infrared emitter on the support element, it is provided according to the preferred specific embodiment that the infrared emitter is detachably held on the support element. This yields multiple advantages. For one, the infrared emitter may be easily replaced, for example, if a defect occurs. For another, this makes it possible for the camera device to be modularly designed so that in certain applications, for example, the camera device may be installed without the infrared emitter and in other applications together with at least one infrared emitter.

It is particularly preferably provided that the support element includes at least two devices for the detachable locking of one infrared emitter each. The devices in this case are advantageously designed identical to one another so that an infrared emitter may be detachably locked on the support element by both the one device as well as by the other device. This further enhances the modularity of the camera device, since it may be decided during assembly whether one or two infrared emitters are to be used. It is understood that more than only two devices may also be provided for the detachable locking on the support element. The devices are situated preferably next to one another. Alternatively, it is also conceivable to provide one device on one side and the other device on the other side of the camera on the support element, in order, for example, to illuminate the driver from two sides.

According to one advantageous refinement of the present invention, it is also provided that the device or the devices are each designed as a plug receptacle or plug receptacles. The infrared emitter may therefore be easily inserted into and locked in the respective plug receptacle. The plug receptacle design makes the assembly particularly simple.

It is preferably provided that each device includes a detent for locking the one or the multiple infrared emitters. The detent may, for example, be elastically deformable or movable detent lugs, which interact with corresponding detent recesses or detent rear grip seats of the infrared emitter or emitters.

According to one advantageous refinement of the present invention, it is provided that the camera device is integratable/integrated into an instrument cluster of a motor vehicle. The instrument cluster is generally always located in the driver's field of vision and is hidden in areas at most by the steering wheel or by an arm of the driver during steering. By providing multiple corresponding camera devices in the instrument cluster, it is possible for the driver to be monitored continuously. Moreover, there are already sufficient options available in the instrument cluster for electrical contacting of the cameral device, so that the integration of the camera device into the instrument cluster is easily implementable. The at least one camera device is situated preferably on the side or in the middle of the instrument cluster. If multiple camera devices are provided, these are then preferably situated at the margins of the instrument cluster, in order to maximize the likelihood of the continuous monitoring of the driver.

It is also preferably provided that the support element is designed as a conductor board. This makes the electrical contacting of camera and infrared emitter particularly easy. The conductor board preferably includes multiple strip conductors, which lead to the device locking the infrared emitter and to the camera unit. If necessary, the detent is designed in such a way that they produce a pre-stressing, via which the infrared emitter is forced with its contact terminals against a strip conductor of the conductor board or against a corresponding contact point of the conductor board, in order to ensure a permanent electrical touch contact. A control unit for operating the camera device may also be situated on the conductor board. However, the conductor board at least includes preferably one contact terminal for the instrument cluster, in order to ensure a simple connection option and integration of the instrument cluster.

According to one advantageous refinement of the present invention, elements are provided, with which the camera device is pluggable, in particular, clippable, into the instrument cluster, in particular, into a conductor board of the instrument cluster. This ensures a particularly simple installation of the camera device, which minimizes the installation effort for the driver monitoring system.

The conductor board of the instrument cluster preferably includes at least one recess for accommodating the camera device and/or the infrared emitter. In this way, a clipping of the camera device directly into the conductor board of the instrument cluster may be implemented.

It is also preferably provided that the infrared emitter includes at least one infrared light-emitting diode (infrared LED). The infrared light-emitting diode has a long service life and is cost-effectively integratable into the driver monitoring system. It is provided, in particular, that the infrared light-emitting diode is directly pluggable into and lockable in the plug receptacle of the device. If necessary, it may also be provided that the plug receptacle is designed for accommodating multiple infrared light-emitting diodes.

According to one alternative specific embodiment, it is preferably provided that only the camera unit is provided on the support element, in particular, on the support element of the camera device insertable into the instrument cluster, whereas the one infrared emitter or multiple infrared emitters are integrated into the instrument cluster separately from the camera device, for example, by situating them on a conductor board of the instrument cluster. In addition to the infrared emitters of the camera device, it is possible to also provide additional infrared emitters outside the instrument cluster, in particular, in the area of the dashboard of the motor vehicle.

The present invention is explained in greater detail below with reference to an exemplary embodiment.

### BRIEF DESCRIPTION OF THE DRAWINGS

FIG. 1 shows a driver monitoring system in a motor vehicle.

FIG. 2 shows a side view of the driver monitoring system.

FIG. 3 shows a top view of the driver monitoring system.

FIG. 4 shows another exemplary embodiment of the driver monitoring system.

FIG. 5 shows another exemplary embodiment of the driver monitoring system.

FIG. 6 shows another exemplary embodiment of the driver monitoring system.

### DETAILED DESCRIPTION OF EXAMPLE EMBODIMENTS

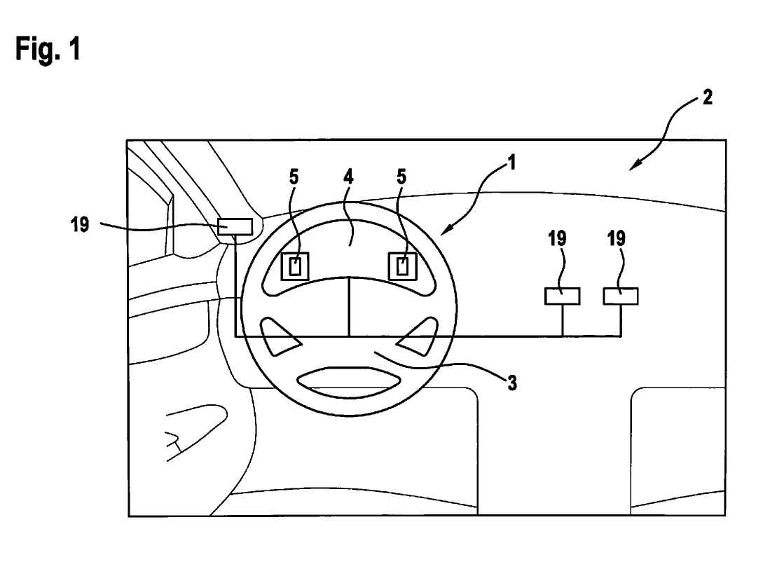

FIG. 1 shows a driver monitoring system 1 for a motor vehicle 2 only suggested here. Motor vehicle 2 includes a steering wheel 3 on the dashboard on the driver's side and behind that an instrument cluster 4 integrated into the dashboard. Instrument cluster 4 is used to display instantaneous condition values of the motor vehicle such as for example, of the instantaneous velocity, of the rotational speed of a drive unit or other information. The displays in this case are situated, in particular, on a conductor board 6 of instrument cluster 4. Driver monitoring system 1 in the present case is integrated into the dashboard and, in particular, into instrument cluster 4. Also provided in instrument cluster 4 are two camera devices 5, which are located at the side marginal areas of instrument cluster 4.

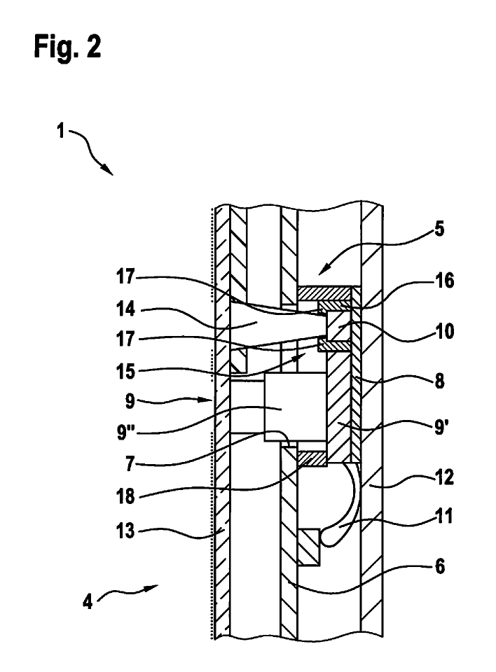

FIG. 2 shows a detailed view of one of camera devices 5 of driver monitoring system 1. FIG. 2 in this case shows a sectional representation through instrument cluster 4, which includes aforementioned conductor board 6. Conductor board 6 includes a recess 7, into which camera device 5 is inserted.

Camera device 5 includes a support element 8, on which a camera unit 9 including a camera chip 9′ and optics 9″ such as, for example, an optic lens and an infrared emitter 10, are situated. Support element 8 is designed as a conductor board, which electrically contacts the infrared emitter and camera unit 9. A ribbon cable 11, which leads to conductor board 6 of instrument cluster 4 and, as a result, electrically connects camera device 5 with instrument cluster 4, also exits the conductor board or support element 8. Support element 8 in this case rests against a rear side of housing wall 12 of instrument cluster where it is clipped, glued or screwed in place. On the side opposite housing wall 12, instrument cluster 4 and, therefore, also camera device 5 is covered by a glass cover 13, which has, in particular, an anti-reflection coating. The anti-reflection coating is preferably discontinued in the area of infrared emitter 10 and of camera unit 9.

Optics 9″ of camera unit 9 of camera device 5 extend through recess 7 to glass cover 13. A light well 14 of the infrared emitter also extends up to glass cover 13.

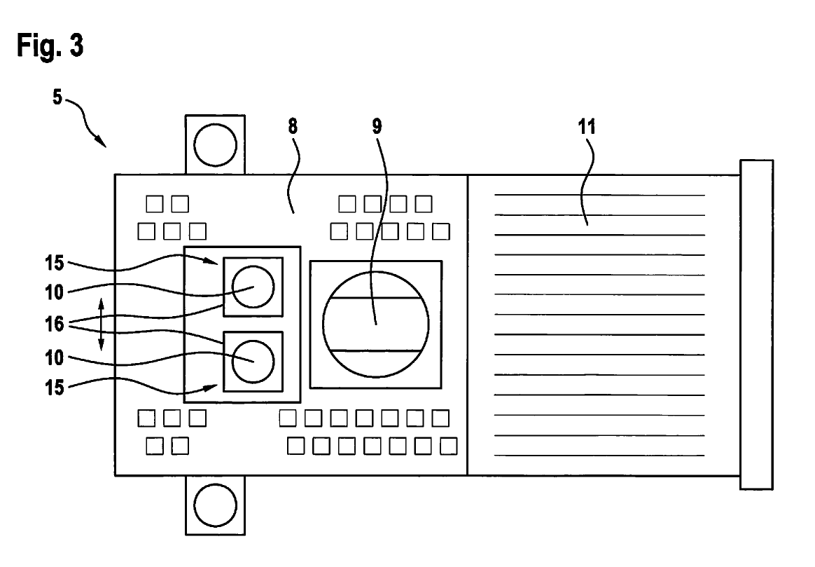

FIG. 3 shows a top view of camera device 5. Camera unit 9 and two of the infrared emitters 10 are situated on support element 8. Infrared emitters 10 in this case are provided next to one another and spaced apart from camera unit 9. For each of infrared emitters 10, support element 8 includes a device 15 for locking the emitters on support element 8. Devices 15 in this case are designed as plug receptacles 16, into which the respective infrared emitter 10 is pluggable for its locking. For this purpose, plug receptacles 16 preferably include detent lugs 17, as they are indicated in FIG. 2 in order to provide a detachable locking for each infrared emitter 10. It may be optionally provided that a device for the detachable locking of camera unit 9 is also provided for camera unit 9 on support element 8. By designing devices 15 as plug receptacles 16, the electrical contacting of infrared emitters 10 is automatically established during installation.

By designing devices 15 for the detachable locking of infrared emitters 10, it is possible to modularly design or vary the number of infrared emitters used. In particular, it is possible thereby to provide fewer than the maximum number of possible infrared emitters 10 on camera device 5. A corresponding number of infrared emitters 10 may be situated on support element 8 as a function of the number of devices 15 or plug receptacles 15. Infrared emitter 10 preferably includes one or multiple infrared light-emitting diodes, which are controllable particularly preferably independently of one another.

Detents 18, which are provided on conductor board 6 of instrument cluster 4 and are used for the detachable locking of the respective camera device 5, are preferably assigned to each camera device 5 as shown in FIG. 2. The detents are depicted in simplified form in FIG. 2. For the design of driver monitoring system 1 according to FIG. 1, two recesses 7 in conductor board 6 of instrument cluster 4 are accordingly needed at the appropriate points. Additional infrared emitters 19, as indicated in FIG. 1, may also be integrated into the dashboard of motor vehicle 2 outside of instrument cluster 4. Infrared emitters 10, 19 and camera units 9 of camera devices 5 are always aligned to the position of the driver or to the driver's head. The infrared emitters emit infrared light, which reflects off the driver and is detected by camera units 9. This makes it possible to monitor the driver even under dark surrounding conditions, without disturbing the driver. As a result, it is possible with driver monitoring system 1 to carry out, for example, a face recognition, tracking of the head, watching of the eyes for determining fatigue or the like.

The installation of driver monitoring system 1 is particularly simple as a result of the design of each camera device 5 as a camera module integratable into instrument cluster 4, as described above. Moreover, the variability of driver monitoring system 1 is enhanced by devices 15, so that the camera module in different specific embodiments is insertable into instrument cluster 4 or into a corresponding instrument cluster, the number of infrared emitters 10, for example, being changed as a function of the respective application, for example, as a function of the respective motor vehicle type. It is equally conceivable to use the camera module or each camera device 5 at another location, for example, for surroundings sensors. It is conceivable, in particular, to also utilize camera device 5 without infrared emitter 10 for surroundings sensors. This results, therefore in a universally applicable camera device 5, in particular, for automotive engineering.

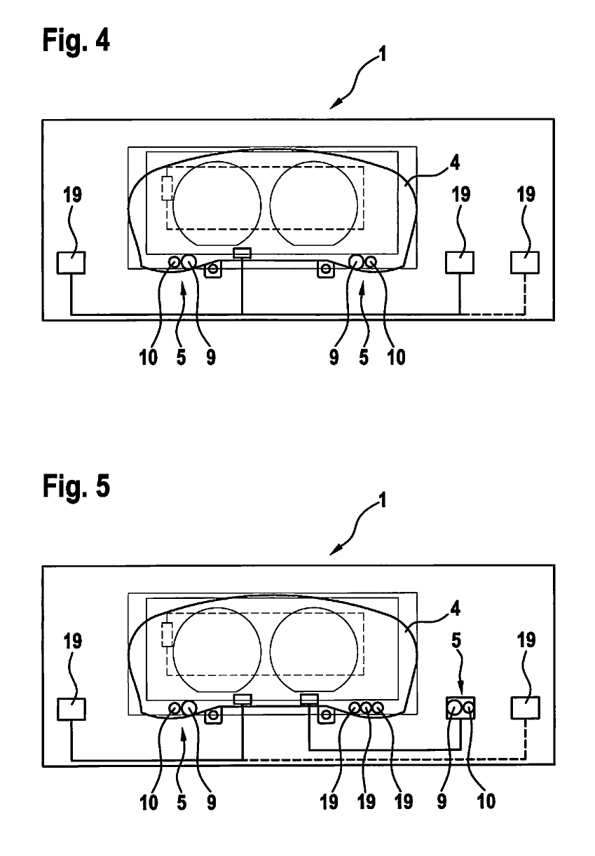

FIGS. 4 and 5 show by way of example different arrangements of driver monitoring system 1, two camera devices 5 being provided in the lower area of an instrument cluster 4 according to FIG. 4. FIG. 5 shows an alternative exemplary embodiment, in which only one camera device 5 is integrated into instrument cluster 4, whereas a second camera device 5 is inserted into the dashboard spaced apart from instrument cluster 4. Hence, different configurations of driver monitoring system 1 are possible. Designing camera device 5 in the form of a variable module allows it to be used in many cases and to be adapted due to its variability.

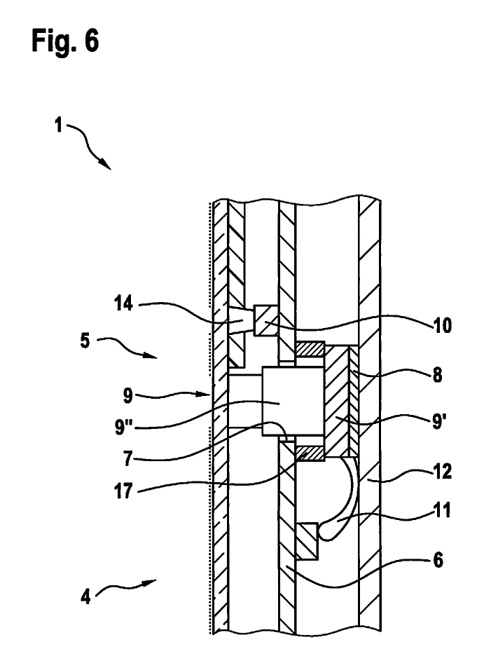

FIG. 6 shows an alternative specific embodiment of driver monitoring system 1, in which support element 8 of camera device 5 is used only for accommodating camera 9, but not for accommodating infrared emitter 10. In this case, therefore, infrared emitter 14 may be situated detached from camera 9. It is provided, in particular, according to the present specific embodiment, that infrared emitter 14 is situated directly on conductor board 6 of instrument cluster 4. Camera 9 is electrically connected to instrument cluster 4 via the connection of ribbon cable 11, infrared emitter 10 being connected directly via conductor board 6, for example, to a microprocessor or the like situated on conductor board 6, which evaluates the data detected by camera 9 with the aid of infrared emitter 10. In this case, therefore, camera device 5 is made up of two module components, which are introducible independently of one another into instrument cluster 4 or the dashboard of motor vehicle 2. In this case, previously described detent 18 is assigned to at least support element 8 and camera unit 9. Infrared emitter 10 in this case is preferably soldered directly to conductor board 6 or detachably locked there by a corresponding plug receptacle, as was previously described. Multiple infrared emitters 10 may, of course, be fastened to conductor board 6 in this way. If conductor board 10 includes a corresponding number of prepared plug receptacles 16, then the positioning and number of desired infrared emitters 10 is also variable.

In the exemplary embodiments described above, it is also possible, of course, to dispense with the additional optics, such as light well 14, assigned to each infrared emitter 10, or to provide correspondingly different optics. Ribbon cable 11 is preferably connected on the one hand to support element 8 via plug elements and, as a result, to the components located thereon, and to conductor board 6 on the other hand.

1-10. (canceled)

11. A driver monitoring system for a motor vehicle, comprising:
at least one camera device which has at least one camera unit situated on a support element and at least one infrared emitter;
wherein the support element includes at least one device for locking the infrared emitter in place on the support element.

12. The driver monitoring system as recited in claim 11, wherein the device is designed for detachable locking of the infrared emitter.

13. The driver monitoring system as recited in claim 11, wherein the support element includes at least two devices for detachable locking of one infrared emitter each.

14. The driver monitoring system as recited in claim 11, wherein each device is a plug receptacle.

15. The driver monitoring system as recited in claim 11, wherein each device includes a detent to lock the infrared emitter or emitters in place.

16. The driver monitoring system as recited in claim 11, wherein the camera device is one of integratable and integrated into an instrument cluster of the motor vehicle.

17. The driver monitoring system as recited in claim 11, wherein the support element is a conductor board.

18. The driver monitoring system as recited in claim 11, further comprising an element with which the camera device is clippable, into a conductor board of the instrument cluster.

19. The driver monitoring system as recited in claim 18, wherein the conductor board of the instrument cluster includes at least one recess for accommodating the camera device.

20. The driver monitoring system as recited in claim 11, wherein the infrared emitter includes at least one infrared light-emitting diode.
